(19) United States
(12) Patent Application Publication
(10) Pub. No.: US 2010/0275718 A1
(43) Pub. Date: Nov. 4, 2010

(54) MANIPULATOR

(75) Inventors: J. Michael Stuart, Rio Rancho, NM (US); Joel N. Beer, Albuquerque, NM (US); David G. Bowling, Los Ranchos, NM (US); John S. Ketchel, Albuquerque, NM (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: MicroDexterity Systems, Inc., Albuquerque, NM (US)

(21) Appl. No.: 12/432,344

(22) Filed: Apr. 29, 2009

(51) Int. Cl. B25J 11/00; B25J 18/00; B25J 17/00; A61B 19/00

(52) U.S. Cl. 74/490.01

(57) ABSTRACT

A manipulator, such as for use in medical procedures, is provided. The manipulator includes a body and a first actuator system connected to the body at a first attachment point and is capable of moving the first attachment point with at least three degrees of freedom. A second actuator system is connected to the body at a second attachment point and is capable of moving the second attachment point with at least three degrees of freedom. A third actuator system is connected to the body at a third attachment point and is capable of moving the third attachment point with at least one degree of freedom.

### BACKGROUND OF THE INVENTION

Conventional devices which are used to perform very complex and/or physically demanding surgical procedures like neurosurgery, spine surgery, ear surgery, head and neck surgery, hand surgery and minimally invasive surgical procedures have a number of drawbacks as it relates to the dexterity of the surgeon. For example, the surgeon can easily become fatigued by the need to manually support the surgical device during its use. Additionally, the surgeon may have to orient his hands in an awkward position in order to operate the device. Furthermore, conventional devices used in such surgical procedures can produce angular magnification of errors. As a result, a surgeon has considerably less dexterity and precision when performing an operation with such surgical devices than when performing an operation by traditional techniques in which the surgeon grasps a tool directly.

Accordingly, there is an increasing interest in the use of powered manipulators, such as robotic and master-slave manipulators for supporting and manipulating surgical tools during medical procedures. Such manipulators can provide a number of advantages to both patients and medical practitioners. In particular, a master/slave controlled manipulator can enhance the dexterity of the surgeon/operator so as to allow the surgeon to manipulate a medical tool with greater dexterity than he could if he was actually holding the tool in his hands. A manipulator can also reduce the fatigue experienced by a surgeon, since it eliminates the need for the surgeon to physically support the medical tool or device during its use. Additionally, the surgeon can let go of the manipulator and perform other tasks without the medical tool undergoing movement, which increases the efficiency of the surgeon and can reduce the number of individuals that are necessary to perform a particular procedure. Thus, manipulators can allow medical procedures to be performed much more rapidly, resulting in less stress on the patient.

However, many manipulators, including those having six degrees of freedom, have some drawbacks in that, in certain orientations, the amount of torque that the manipulator can apply is limited. This restricts the work that can be done by the manipulator in such orientations. Moreover, some manipulators have singularity points within their operational envelopes. At these singularity points, two or more manipulator joints become redundant and fewer degrees of the freedom can be exercised. This can cause a manipulator mechanism to become locked or impeded such that it can no longer move freely.

### BRIEF SUMMARY OF THE INVENTION

The invention provides a manipulator, such as for use in medical procedures. The manipulator includes a body and a first actuator system connected to the body at a first attachment point and is capable of moving the first attachment point with at least three degrees of freedom. A second actuator system is connected to the body at a second attachment point and is capable of moving the second attachment point with at least three degrees of freedom. A third actuator system is connected to the body at a third attachment point and is capable of moving the third attachment point with at least one degree of freedom.

### BRIEF DESCRIPTION OF THE SEVERAL VIEWS OF THE DRAWINGS

FIG. 1 is a schematic view of an exemplary manipulator according to the invention that includes three three degree of freedom linear axis serial actuators.

FIG. 2 is a schematic view of an alternative embodiment of a manipulator according to the invention that includes three three degree of freedom rotary axis serial actuators.

FIG. 3 is a schematic view of another alternative embodiment of a manipulator according to the invention that includes three three degree of freedom linear axis parallel actuators.

FIG. 4 is a schematic view of a further alternative embodiment of a manipulator according to the invention that includes a three degree of freedom linear axis parallel actuator and two three degree of freedom linear axis serial actuators.

FIG. 5 is a schematic view of an alternative embodiment of a manipulator according to the invention that includes two three degree of freedom rotary axis serial actuators and a three degree of freedom rotary axis parallel actuator.

FIG. 6 is a schematic view of a further alternative embodiment of a manipulator according to the invention that includes two three degree of freedom mixed architecture serial actuator and a three degree of freedom mixed architecture parallel actuator.

FIG. 7 is a schematic view of a further alternative embodiment of a manipulator according to the invention that includes two three degree of freedom linear axis serial actuators and a third two degree of freedom linear axis serial actuator.

FIG. 8 is a schematic view of a further alternative embodiment of a manipulator according to the invention that includes two three degree of freedom linear axis serial actuators and a third one degree of freedom linear actuator.

### DETAILED DESCRIPTION OF THE INVENTION

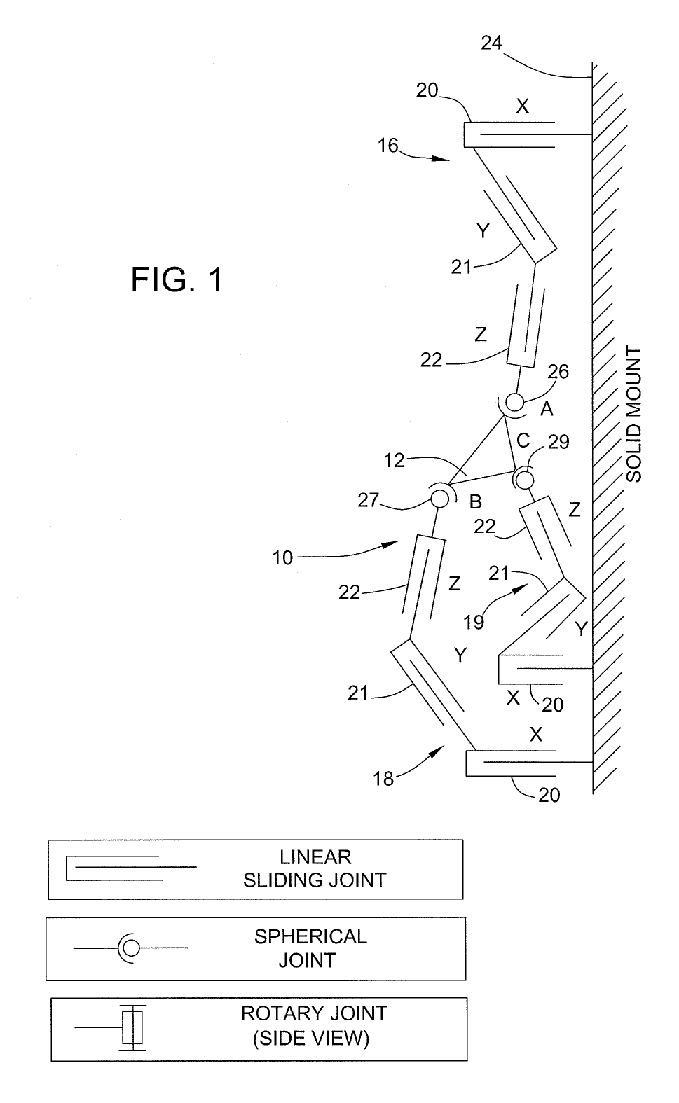

Referring now more particularly to FIG. 1 of the drawings, there is shown an illustrative embodiment of a manipulator constructed in accordance with the present invention. The illustrated manipulator 10 can interchangeably support and move a body with six degrees of freedom. In this case, the moving body can comprise a support member 12 that carries an end effector, e.g. a medical tool holder or mount 14. For ease of reference, the support member 12 in the illustrated embodiment has a triangular configuration. However, as will be appreciated, the invention is not limited to any particular type, form or shape of moving body. In this regard, the invention is also not limited to any particular type of medical tool, tool holder or support structure rather any suitable tool and/or tool support can be used with the manipulator including, but not limited to, needle holders, staple or clamp appliers, probes, scissors, forceps, cautery, suction cutters, dissectors, drills, saws, lasers, ultrasonic devices and diagnostic devices. The tools can be reusable, limited reuse or disposable. If the medical tool has moving parts that are conventionally human powered, the manipulator 10 can be adapted to accommodate an actuator dedicated to powering the tool such as for example an electric, pneumatic or hydraulic actuator.

While the present invention is described in connection with performing complex medical procedures, the manipulator of the present invention is not limited to such applications. Rather, the manipulator of the present invention can be used in any application involving dexterous tasks. For example, it can be used in applications involving the remote manipulation of hazardous materials. It can also be used in complex assembly or repair operations to perform autonomous, but repetitive, tasks normally done by humans.

In order to provide dexterity enhancement for an operator/surgeon in performing surgical and certain interventional radiology procedures, the manipulator 10 can be used as a slave robot in a master-slave robotic system. The manipulator 10 can also be used as a master robot in such a system. In a master-slave robotic system, a surgeon/operator provides position input signals to the “slave” manipulator via a master or haptic interface which operates through a controller or control console. Specifically, with the manipulator 10 of the present invention serving as the slave robot, the surgeon indicates the desired movement of the tool held by the manipulator 10 through the use of an input device on the haptic interface such as a six degree of freedom tool handle with or without force feedback, joystick, foot pedal or the like. The haptic interface relays these signals to the controller, which, in turn, applies various desired predetermined adjustments to the signals prior to relaying them to the slave manipulator. Any haptic interface having an six or more degrees of freedom (DOF) can be used to control the manipulator 10 via the controller. Examples of haptic interfaces or masters which can be used with the present invention include the Freedom 6S available from MPB Technologies of Montreal, Canada, and other haptic interfaces commercially available from Sensable Technology of Cambridge, Mass. and MicroDexterity Systems of Albuquerque, N. Mex.

Based on the signals provided by the controller, the manipulator 10 executes the desired movement or operation of the tool. Thus, any desired dexterity enhancement can be achieved by setting up the controller to perform the appropriate adjustments to the signals sent from the haptic interface. For example, this can be accomplished by providing the controller with software which performs a desired dexterity enhancement algorithm. Software dexterity enhancement algorithms can include position scaling (typically downscaling), force scaling (up-scaling for bone and cartilage, downscaling for soft tissue), tremor filtering, gravity compensation, programmable position boundaries, motion compensation for tissue that is moving, velocity limits (e.g., preventing rapid movement into brain, nerve or spinal cord tissue after drilling through bone), and, as discussed in greater detail below, image referencing. These and other examples of possible algorithms are well known in the field of robotics and described in detail in published literature. The ZMP SynqNet® Series Motion Controllers which employ the SynqNet system and are available from Motion Engineering of Santa Barbara, Calif. are one example of a suitable controller for use with the present invention (see www.synqnet.org and www.motioneng.com). Another example of a suitable controller is the Turbo PMAC available from Delta Tau Data Systems of Northridge, Calif.

To effect movement of the support member 12 in space, the manipulator 10 includes first and second separate, independent three degree of freedom actuator systems 16, 18 each of which connects to the support member 12 at a respective attachment point. The manipulator further includes a separate third actuator system 19 that is at least one degree of freedom and attaches to the support member 12 at a separate third attachment point that is coplanar with the attachment points of the first and second actuator systems. The first, second and third actuator systems 16, 18, 19 are each supported on a solid mount. The first and second actuator systems 16, 18 can be any type of three degree of freedom actuator system. Likewise, the third actuator system 19 can be any type of actuator system that provides the desired degrees of freedom, although an actuator system with three or more degrees of freedom actuator is presently preferred. The use of a three degree of freedom actuator provides the manipulator with a total of nine degrees of freedom. One example of such a manipulator is shown in FIG. 1.

As will be appreciated by those skilled in the art, six degrees of freedom are all that is required to define the position of the support member in space. Thus, the seventh, eighth and ninth degrees of freedom provided by the preferred embodiment of the invention are redundant degrees of freedom. The redundant degrees of freedom provide for good torque delivery in a wider variety of orientations as compared to a manipulator having just six degrees of freedom (i.e., no redundant degrees of freedom) and expands the operational envelope beyond what many six degree of freedom manipulators can achieve. In particular, the range of motion of all hybrid serial/parallel mechanisms is defined by a series of singularity points where the manipulator becomes locked and can no longer move freely. For example, these singularity points can happen when the manipulator is at full extension with the mechanical elements of the manipulator binding against one another in such a way that the manipulator cannot provide enough force or torque to move itself and whatever tool is being manipulated. The redundant degrees of freedom help solve this problem.

Various embodiments of seven degree of freedom manipulator systems in which the seventh degree of freedom is provided by a third actuator system that is integrated with the moving body are disclosed in commonly owned U.S. patent application Ser. No. 11/710,023 filed Feb. 23, 2007, the disclosure of which is incorporated herein by reference. As compared to those systems, the arrangement of the present invention provides the advantage that full six degree of freedom movement can be delivered directly at the moving body rather than through the use of the third actuator system attached to the moving body. In particular, as noted above, the arrangement of the present invention provides a third actuator system 19 that attaches to the support member 12 at a separate third attachment point rather than being integrated into the support member 12. This means that the arrangement of the present invention moves the heavy motors and reduction mechanisms associated with the third actuator system to a point away from the moving body. In most cases, this will advantageously reduce the inertial forces associated with the manipulator.

Conventional platform manipulators typically use six actuators each of which connects to the platform at a different attachment point. The actuators can have various configurations. Platform manipulators can experience problems when the platform is moved into a position in which the actuator link and the attachment point on the platform are, or are nearly, coplanar. In such a position, the actuator becomes useless and the manipulator can lock up. This is because the actuator can only push or rotate, but not both. When you replace the single degree of freedom actuators with three three degree of freedom actuators as in the preferred embodiment of the invention, you have the ability to generate any force vector in a three degree of freedom working space. For example, the three degree of freedom manipulators can be operated together to produce translation or rotation of the support member. Even if the attachment points and the actuator become coplanar, the actuator can produce a force vector that will still control the position of the attachment point. As a result, the manipulator of the present invention is much more dexterous and has fewer singularity points within the workspace than conventional six degree of freedom platform manipulators. The singularity points with the preferred nine degree of freedom embodiment are mostly defined by the points where actuator links are touching or where joints/links have reached a limit of their movement. Additionally, the extra degrees of freedom allow for a reduction or modulation of the motor power associated with the manipulator leading to better control of power consumption and heat.

In the FIG. 1 embodiment, each of the first, second and third actuator systems 16, 18, 19 comprises a simple linear axis serial actuator. In particular each actuator system includes three linear sliding joints or actuators 20, 21, 22. Each of the sliding joints/actuators 20, 21, 22 translates or slides along a respective Cartesian coordinate axis, i.e. x, y or z. In this case, each of the actuator systems includes an x-axis linear joint/actuator 20 that has one end connected to a solid mount 24 and a second end connected to a y-axis linear joint/actuator 21. The opposite end of the y-axis linear joint/actuator 21 is, in turn, connected to a z-axis linear joint/actuator 22. The z-axis linear joint/actuator 22 of each of the first, second and third actuator systems 16, 18, 19 connects at the respective attachment point to the support member 12.

In the embodiment illustrated in FIG. 1, the attachment points of the first and second actuator systems each comprise a joint 26, 27, such as a spherical joint or its equivalent, having three rotary degrees of freedom. Because of the two spherical joints 26, 27, the support member 12 would be incompletely constrained in the absence of the third actuator system. In particular, the support member 12 would be free to rotate about a line connecting the centers of the two spherical joints 26, 27. The third actuator system 19 constrains this free motion by providing at least a one degree of freedom actuator, and in this case a three degree of freedom actuator, that connects to a third point on the support member 12 that is in a plane defined by the centers of the two spherical joints 26, 27 and the line connecting the centers. The attachment of the third actuator system 19 to the support member 12 also can be via a third three degree of freedom rotary joint 29 such as a spherical joint or its equivalent. The joints can have any desired construction that provides the necessary degrees of rotary freedom. Moreover, single joints at the attachment points can be replaced with multiple joints that collectively provide equivalent degrees of freedom.

For sensing the positions of the various rotary joints 26, 27, 29 and, in turn, the support member 12 and/or tool mount, all or some of the rotary joints can be equipped with position sensors. Each of the drive systems of the manipulator can be in communication with the controller and the position sensors can provide position information in a feedback loop to the controller. It will be appreciated that any number of different conventional position sensors can be used such as, for example, optical encoders. Moreover, the various drive systems can also be equipped with force sensors for sensing the forces or torques applied by the actuators so as to enable a determination of the forces and torques applied to the support member and/or the tool mount. This information can again be provided in a feedback control loop to the controller, for example to allow force feedback to the input device of a haptic interface. Of course, any known method for measuring forces and/or torques can be used, including, for example, foil type or semiconductor strain gauges or load cells.

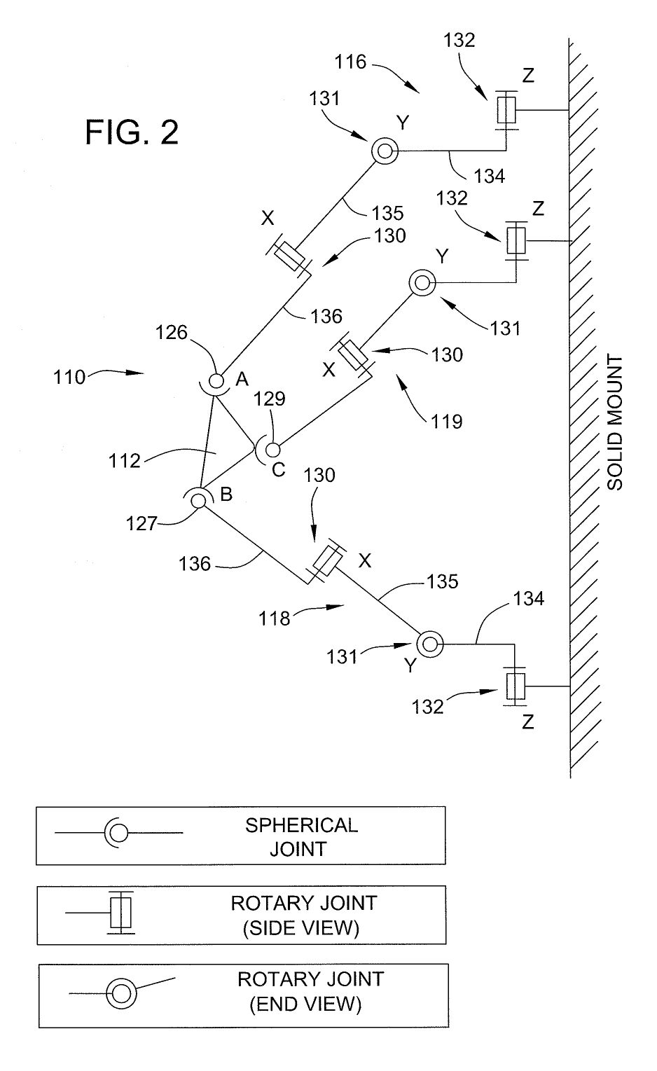

As noted above, the first, second and third actuator systems 16, 18 can be any type of three or more degree of freedom actuator systems. For example, an alternative embodiment in which three rotary joint/actuators 130, 131, 132 are employed in the first, second and third actuator systems 116, 118, 119 as opposed to linear joints/actuators is shown in FIG. 2. In the embodiment of FIG. 2, elements similar to those found in the FIG. 1 embodiment are given corresponding reference numbers in the 100s. As the case with the FIG. 1 embodiment, the rotary joint/actuators 130, 131, 132 are in a serial arrangement with each rotary joint/actuator rotating about a respective Cartesian coordinate axis, i.e. x, y or z. In the arrangement illustrated in FIG. 2, each of the first, second and third actuator systems 116, 118 includes a z-axis rotary joint/actuator 132 that is connected to a solid mount 124. The output shaft of the z-axis rotary joint/actuator 132 is connected to a first link 134 that extends to a y-axis rotary joint/actuator 131. The output shaft of the y-axis rotary joint/actuator 131, in turn, connects via a second link 135 to a x-axis rotary joint/actuator 130, which has an output shaft that connects to a third link 136 that connects at a respective attachment point to the support member 112. With each actuator system 116, 118, 119 the angles of the three rotary joints/actuators 130, 131, 132 define the positions of the respective attachment points. The attachment points, in this case, comprise three degree of rotary freedom spherical joints 126, 127.

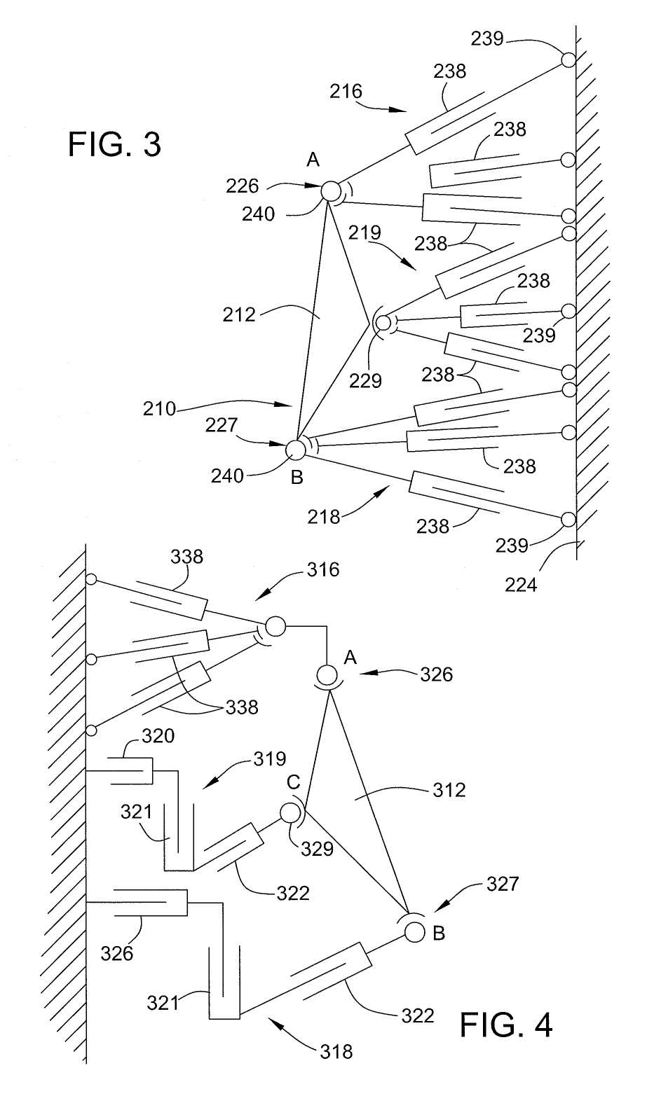

A further embodiment that employs three degree of freedom parallel, as opposed to serial, actuators as the first, second and third actuator systems is shown in FIG. 3. In FIG. 3, elements similar to those found in the FIGS. 1 and 2 embodiments are given corresponding reference numbers in the 200s. Specifically, in the FIG. 3 embodiment, each of the first, second and third actuator systems 216, 218, 219 comprises three linear joints/actuators 238 arranged in parallel. Each of the linear joints/actuators 238 is connected at one end to a solid mount 224 via a respective three degree of rotary freedom spherical joint 239. The other end of each of the linear joint actuators 238 is connected to a fixed sphere 240 so as to form a tripod arrangement in which the tip, i.e. the fixed sphere, can be moved in space. The fixed sphere is part of a spherical joint 226, 227, 229 that defines the attachment point to the support member 212. In this case, one of the three linear joint actuators 238 of each actuator system 216, 218, 219 is rigidly connected to the fixed sphere while the other two are connected to the sphere in such a way that they each can rotate about the sphere with three degrees of freedom.

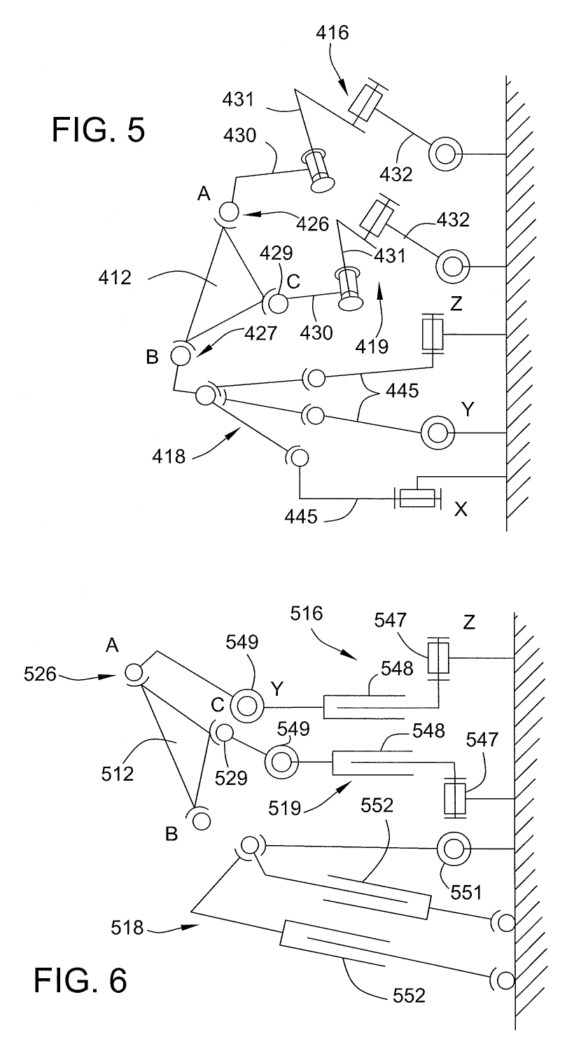

As shown in FIGS. 4-5, the first, second and third actuator systems can have different configurations. More specifically, in the embodiment of the invention shown in FIG. 4, the first actuator system 316 comprises a three degree of freedom parallel linear actuator having a tripod configuration like that used for the first and second actuator systems in the embodiment of FIG. 3. In FIG. 4, elements similar to those found in the embodiments of FIGS. 1-3 are given corresponding reference numbers in the 300s. In the FIG. 4 embodiment, the second and third actuator systems 318, 319 comprise three degree of freedom serial linear actuators (with linear actuators 320, 321, 322) like that used in the embodiment of FIG. 1. Again, each of the actuator systems 316, 318, 319 connects to the support member 312 at a respective attachment point comprising a spherical joint 326, 327, 329.

In the embodiment of FIG. 5, elements similar to those found in the embodiments of FIGS. 1-4 are given corresponding reference numbers in the 400s. In FIG. 5, the first and third actuator systems 416, 419 comprise three degree of freedom serial rotary actuators (with rotary actuators 430, 431, 432) like that used in the embodiment of FIG. 2 and the second actuator system 418 comprises a three degree of freedom parallel rotary actuator, which is generally similar to the three degree of freedom parallel tripod actuators of FIG. 3 but with rotary joints/actuators 445 instead of linear joints/actuators. In particular, the three degree of freedom parallel rotary second actuator system 418 includes three legs each of which is connected to a respective rotary joint/actuator 445. Each rotary joint/actuator 445 is connected to the solid mount 424 and rotates about a respective one of the Cartesian coordinate axes, i.e. x, y and z. Again, each of the actuator systems 416, 418, 419 connects to the support member 412 at a respective attachment point comprising a spherical joint 426, 427, 429.

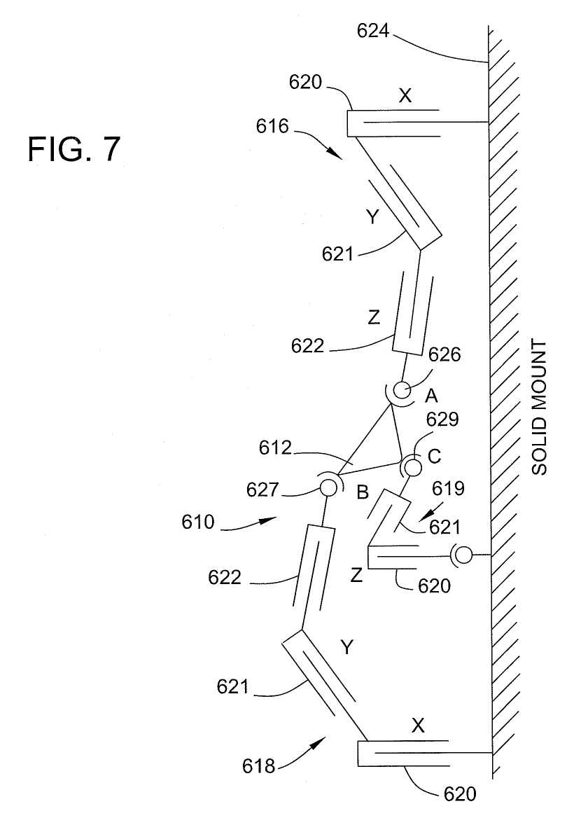

As shown in the embodiment of FIG. 6, the individual first, second and third actuator systems 516, 518, 519 can have mixed architectures including both linear and rotary joints/actuators. In FIG. 6, elements similar to those found in the embodiments of FIGS. 1-5 are given corresponding reference numbers in the 500s. In the FIG. 6 embodiment, the first and third actuator systems 516, 519 are serial arrangements that include a z-axis rotary joint/actuator 547 having an output shaft connected to a link that connects to a linear joint/actuator 548 that, in turn, is connected to a y-axis rotary joint/actuator 549. The second actuator system 518 is a parallel tripod arrangement consisting of one leg with a rotary joint/actuator 551 and two legs with linear joints/actuators 552. Again, each of the actuator systems 516, 518, 519 connects to the support member 512 at a respective attachment point comprising a spherical joint 526, 527, 529. The FIG. 6 embodiment illustrates that any combination of rotary and linear actuators that provides the desired three or more degrees of freedom can be used to form the first, second and third actuator systems.

While the nine degree of freedom arrangements of FIGS. 1-6 offer comparatively fewer singularity points, the manipulator could also be configured with seven or eight degrees of freedom by configuring one of the first, second and third actuator systems with one or two degrees of freedom. For example, in the FIG. 7 embodiment, each of the first and second actuator systems 616, 618 comprises a simple linear axis three degree of freedom serial actuator with three linear joints/actuators 620, 621, 622 similar to the embodiment of FIG. 1. The third actuator system 619 of the FIG. 7 embodiment, in turn, comprises a two degree of freedom actuator with, in this case, two linear actuators 620, 621 arranged in series having one end connected to the solid mount 624 and a second end connected to the support member 612. Again, all the attachment points comprise respective spherical joints 626, 627, 629. The manipulator thus has a total of eight degrees of freedom.

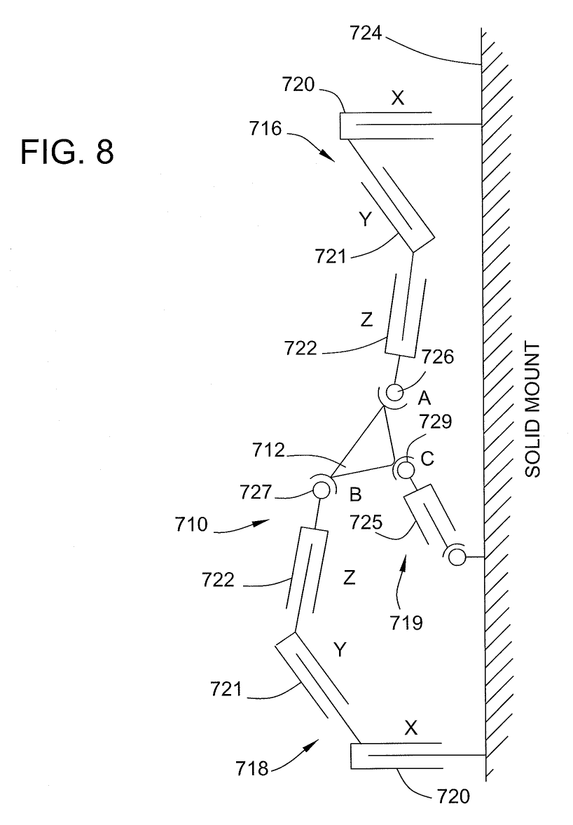

A manipulator with a total of seven degrees of freedom is shown in FIG. 8. In the embodiment of FIG. 8, the first and second actuator systems 716, 718 each comprise a simple linear axis three degree of freedom serial actuator with three linear joints/actuators 720, 721, 722 similar to the embodiment of FIG. 1. The third actuator system 719, in turn, comprises a one degree of freedom actuator with, in this case, a single linear actuator 725 having one end connected to the solid mount 724 and a second end connected to the support member 712. The attachment points to the support member again comprise three degree of freedom spherical joints 726, 727, 729.

In view of the foregoing, it will be appreciated that the present invention provides a manipulator that provides up to nine or more degrees of freedom. The redundant degrees of freedom provide improved performance by improving torque delivery in certain orientations and by helping to eliminate certain singularity points. Manipulators having first, second and third actuator systems with particular configurations are shown in the drawings and described herein. Of course, other types of three degree of freedom actuator systems could also be used for the first, second and third actuator systems. For example, one or more of the actuator systems could be based on a so-called r-theta mechanism, which is a two degree of freedom radial coordinate engine. A further actuator can then be connected to each r-theta mechanism which is able to independently move the corresponding r-theta mechanism out of its respective rotational plane. The result is that the actuator systems comprise independent three degree of freedom actuator systems. Other arrangements are also possible.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A manipulator comprising:
a body;
a first actuator system connected to the body at a first attachment point and capable of moving the first attachment point with at least three degrees of freedom;
a second actuator system connected to the body at a second attachment point and capable of moving the second attachment point with at least three degrees of freedom; and
a third actuator system connected to the body at a third attachment point and capable of moving the third attachment point with at least one degree of freedom.

2. The manipulator of claim 1 wherein the first, second and third attachment points are arranged in a common plane.

3. The manipulator of claim 1 wherein the first, second and third actuator systems are respectively supported on solid mounts.

4. The manipulator of claim 1 wherein the first and second attachment points each comprises a respective three degree of freedom joint.

5. The manipulator of claim 1 wherein the third attachment point comprises a three degree of freedom joint.

6. The manipulator of claim 1 wherein the third actuator system is capable of moving the third attachment point with at least three degrees of freedom.

7. The manipulator of claim 1 wherein the third actuator system is capable of moving the third attachment point with at least two degrees of freedom.

8. The manipulator of claim 1 wherein each of the first, second and third actuator systems comprises a serial actuator system.

9. The manipulator of claim 8 wherein each of the first, second and third actuator systems employs at least three linear joints/actuators.

10. The manipulator of claim 8 wherein each of the first, second and third actuator systems employs at least three rotary joints/actuators.

11. The manipulator of claim 8 wherein at least one of the first, second and third actuator systems employs a combination of rotary and linear joints/actuators.

12. The manipulator of claim 1 wherein the first, second and third actuator systems comprise parallel actuator systems.

13. The manipulator of claim 12 wherein each of the first, second and third actuator systems employs at least three linear joints/actuators.

14. The manipulator of claim 12 wherein each of the first, second and third actuator systems employs at least three rotary joints/actuators.

15. The manipulator of claim 12 wherein at least one of the first, second and third actuator systems employs a combination of rotary and linear joints/actuators.

16. The manipulator of claim 1 wherein the first actuator system comprises a serial actuator system and the second actuator system comprises a parallel actuator system.

17. The manipulator of claim 1 wherein the body includes a tool mount.

18. The manipulator of claim 1 wherein the body comprises a plate.
